(19) United States
(12) Patent Application Publication
(10) Pub. No.: US 2024/0190825 A1
(43) Pub. Date: Jun. 13, 2024

(54) PROCESS FOR PREPARATION OF CABOZANTINIB

(71) Applicant: BIOCON LIMITED, Bangalore (IN)

(72) Inventors: Ramakrishna Parameshwar BHAT, Dhareshwar (IN); Narasimman KUMAR, Bangalore (IN); Venu Thirunelimada DEVAIAH, Bangalore (IN); Pratik Rameshchandra PATEL, Bangalore (IN); Ranjeet NAIR, Bangalore (IN); Lankeswararao MATTI, Andhra Pradesh (IN); Jithendrababu Raghunadha CHETTY, Andhra Pradesh (IN)

(21) Appl. No.: 18/283,281

(22) PCT Filed: Mar. 24, 2022
(86) PCT No.: PCT/IB2022/052685

§ 371 Date: Sep. 21, 2023

(30) Foreign Application Priority Data
Mar. 24, 2021 (IN) 202141012881
Jun. 29, 2021 (IN) 202141029133

(51) Int. Cl. C07D 215/233; A61K 9/14; A61K 31/47

(52) U.S. Cl. CPC C07D 215/233; A61K 9/146; A61K 31/47

(57) ABSTRACT

The present invention provides for novel amorphous solid dispersions of form of cabozantinib malate, and a pharmaceutically acceptable excipient and process for the preparation of cabozantinib malate. The invention also provides a novel crystalline polymorph of cabozantinib.

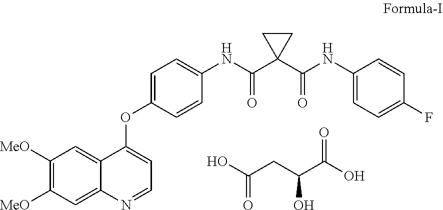

### RELATED APPLICATION

This application claims the benefit of priority of our Indian patent applications IN 202141012881 filed on Mar. 24, 2021, and IN 202141029133 filed on Jun. 29, 2021, which are incorporated herein by reference.

### TECHNICAL FIELD

The present invention refers to an amorphous solid dispersion of form of cabozantinib malate, and a pharmaceutically acceptable excipient and process for the preparation of cabozantinib malate.

### BACKGROUND AND PRIOR ART OF THE DISCLOSURE

CABOMETYX is a kinase inhibitor indicated for the treatment of patients with advanced renal cell carcinoma (RCC) who have received prior antiangiogenic therapy.

CABOMETYX is the (S)-malate salt of cabozantinib, a kinase inhibitor. Cabozantinib (S)-malate is described chemically as N-(4-(6,7-dimethoxyquinolin-4-yloxy)phenyl)-N′-(4fluorophenyl)cyclopropane-1,1-dicarboxamide, (2S)-hydroxybutanedioate. The chemical structure of cabozantinib (S)-malate salt is:

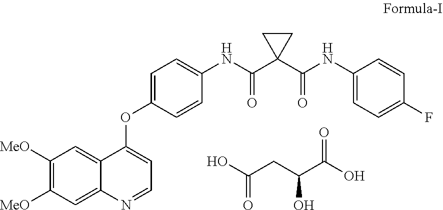

Cabozantinib (S)-malate salt is a white to off-white solid that is practically insoluble in aqueous media.

CABOMETYX (cabozantinib) tablets are supplied as film-coated tablets containing 20 mg, 40 mg, or 60 mg of cabozantinib, which is equivalent to 25 mg, 51 mg, or 76 mg of cabozantinib (S)-malate, respectively.

U.S. Pat. No. 7,579,473 B2 discloses Cabozantinib and its pharmaceutically acceptable salts.

U.S. Pat. No. 8,877,776 B2 discloses Cabozantinib (S)-malate salt and discloses said salt in the crystalline forms (N-1), (N-2) and amorphous and processes of preparation thereof.

U.S Pat. No. 11,091,439 B2 claims Cabozantinib (S)-malate salt, wherein said salt is crystalline.

U.S. Pat. No. 9,815,789 B2, discloses crystalline forms M₁, M₂, M₃ & M₄ of (L)-malate salt of N-(4-(6,7-dimethoxyquinolin-4-yloxy) phenyl)-N′-(4-fluorophenyl)cyclopropane-1,1-dicarboxamide and crystalline forms M₁, M₂ & M₃ of N-(4-(6,7-dimethoxyquinolin-4-yloxy) phenyl)-N′-(4-fluorophenyl)cyclopropane-1, 1-dicarboxamide and processes of preparation thereof.

PCT publication WO 2018104954 A1, discloses crystalline forms M and S of (L)-malate salt of N-(4-(6,7-dimethoxyquinolin-4-yloxy) phenyl)-N′-(4-fluorophenyl)cyclopropane-1,1-dicarboxamide; crystalline forms M, S, N, and R of hydrochloride salt of N-(4-(6,7-dimethoxyquinolin-4-yloxy) phenyl)-N′-(4-fluorophenyl)cyclopropane-1,1-dicarboxamide and a processes of preparation thereof.

CN104961680 A discloses crystal A and crystal B of hydrochloride salt of N-(4-(6,7-dimethoxyquinolin-4-yloxy) phenyl)-N′-(4-fluorophenyl)cyclopropane-1,1-dicarboxamide and process for its preparation.

CN104961681 A discloses various acid addition salts of N-(4-(6,7-dimethoxyquinolin-4-yloxy) phenyl)-N′-(4-fluorophenyl)cyclopropane-1,1-dicarboxamide and process for its preparation.

PCT publication WO 2020057622 A1, discloses crystalline forms CSI and CSIII of Cabozantinib (S)-malate salt.

PCT publication WO 2020075196 A1, discloses crystalline forms C2, C3, C4 and C5 of Cabozantinib (S)-malate salt.

An organic compound may give rise to a variety of solid forms either crystalline or amorphous having distinct physical properties. The variation in the physical properties frequently results in differences in bioavailability, stability, etc.

Some polymorphic forms of drug substances suffer from the drawbacks of spontaneous conversion to other crystalline forms during storage, resulting in concomitant change, not only in the physical form and shape of the drug crystals, but also associated changes in distinct physical properties. Generally, the forms will revert to a more thermodynamically stable form, often a form with lower solubility. Such a thermodynamically stable form may sometimes result in a reduced or suboptimal bioavailability, especially for oral administration. Because of different physical and chemical properties, different crystal forms of the drug may have different dissolution and absorption in the body, which in turn affects the clinical efficacy and safety of the drug to a certain extent. Especially for poorly soluble solid drugs, the crystal form will have a greater impact. Therefore, the crystal form of a drug must be an important content of drug research and an important content of drug quality control.

At present, although there are various disclosures of crystalline forms of Compound of Formula-I, the properties of the reported polymorphs are not yet complete, and suffer from disadvantages. For example, as disclosed in WO2020057622 A1, the PCT publication WO2015177758 A1 discloses the crystalline forms M1, M2, M3 and M4 of compound I, wherein the crystalline form M4 is a better crystal form, but this crystal form also has low solubility, fluidity, compressibility, and resistance. Problems with poor tensile strength and adhesion.

Cabozantinib is a poorly water-soluble drug and belongs to BCS class II compound (as published in the EMA Assessment report). Higher solubility is conducive to improving the absorption of the drug in the human body, increasing the bioavailability, and making the drug play a better therapeutic effect; in addition, the higher solubility can reduce the dose of the drug while ensuring the efficacy of the drug, thereby reducing the drug side effects and improve the safety of medicines.

There remains a continuing need for amorphous form of N-(4-(6,7-dimethoxyquinolin-4-yloxy)phenyl)-N′-(4fluorophenyl)cyclopropane-1,1-dicarboxamide, (2S)-hydroxybutane dioate or its solid dispersions that are stable, but also for processes to produce N-(4-(6,7-dimethoxyquinolin-4-yloxy)phenyl)-N′-(4fluorophenyl) cyclopropane-1,1-dicarboxamide, (2S)-hydroxybutanedioate, which are convenient to scale-up for commercial production quantities and yield both formulation and therapeutic benefits.

PCT publication WO 2012109510 A1 discloses the synthesis of Cabozantinib, it involves the condensation of 4-(6,7-dimethoxy-quinoline-4-yloxy)-phenylamine with 1-(4-Fluoro-phenylcarbamoyl)-cyclopropanecarbonyl chloride in THF & Water as solvent combination to obtain Cabozantinib as shown in the scheme-1.

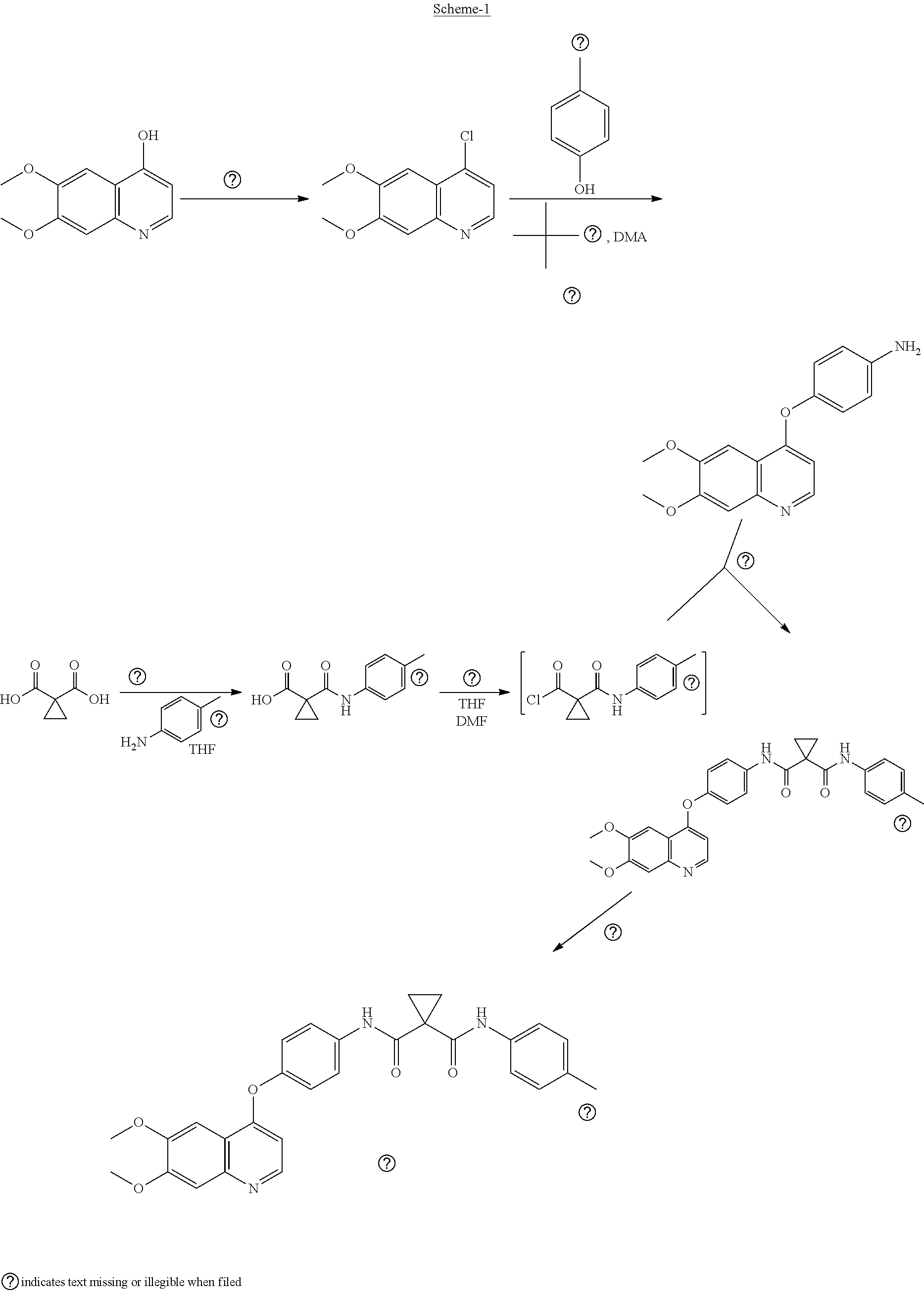

This method involves the usage of oxalyl chloride in the step prior to the step of formation of cabozantinib. Process has disadvantages that it leads to a number of impurities. One such major impurity has been identified, isolated and characterized in the present invention as impurity-1 (N¹,N²-bis(4-((6,7-dimethoxyquinolin-4- yl)oxy)phenyl)oxalamide of structural formula IX). In other words, the use of thionyl chloride or oxalyl chloride, results in significant a yield loss (>15%) due to purification.

Chinese patent publication CN 109836381 A discloses the synthesis of Cabozantinib, it involves the condensation of 4-(6,7-dimethoxy-quinoline-4-yloxy)-phenylamine with 1-(4-Fluoro-phenylcarbamoyl)-cyclopropanecarboxylic acid using condensing agents in presence of an organic base and a polar organic solvent. The condensing agent used were selected from 1-ethyl-(3-dimethylaminopropyl) carbodiimide hydrochloride (EDCI), diethyl cyanophosphate, 2-(7-benzotriazide oxide) Azole)-N,N,N′,N′-tetramethylurea hexafluorophosphate (HATU), O-benzotriazole-tetramethylurea hexafluorophosphate (HBTU), O-benzotriazole-N,N,N′,N′-tetramethylurea tetrafluoroborate (TBTU), N,N-diisopropylcarbodiimide (DIC) and dicyclohexylcarbodiimide (DCC). The organic base is selected from triethylamine, N,N-diisopropylethylamine (DIEA) and 4-dimethylaminopyridine(DMAP). The reaction solvent is preferably one of N,N-dimethylacetamide, N,N-dimethylformamide, N-methylpyrrolidone, tetrahydrofuran, dioxane, dimethylsulfoxide and acetonitrile. This is as shown in the scheme-2.

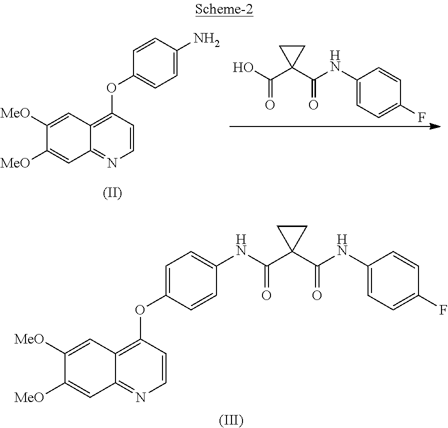

This scheme involves usage of expensive coupling agents which are economically not viable.

Hence, the said process is not suitable for commercial scale. Peptide coupling agents (HATU, HBTU, & HCTU) are potent immune sensitizers. They have caused cases of both skin and respiratory sensitization in the form of rashes and lesions (dermatitis) and coughing, sneezing, and throat-closing (anaphylaxis) reactions. Peptide coupling agents can modify human proteins, which is the most likely mechanism through which they cause immune sensitization (Ref: J. Org. Chem. 2020, 85, 1764-1768)

PCT publication WO 2019234761 A1 discloses the synthesis of Cabozantinib, it involves the condensation of 4-(6,7-dimethoxy-quinoline-4-yloxy)-phenylamine with 1-(4-Fluoro-phenylcarbamoyl)-cyclopropanecarboxylic acid using condensing agents hydroxybenzotriazole (HOBt), 1-ethyl-3-(3-dimethylaminopropyl)carbodiimide hydrochloride (EDCHCI), benzotriazol-1-yl-oxytripyrrolidinophosphonium hexafluorophosphate (PyBOP), Bromo-trispyrrolidino phosphonium hexafluorophosphate (PyBrOP), 0-(benzotriazol-1-yl)-N,N,N′,N′-tetramethyluronium tetrafluoroborate (TBTU), 2-(1H-Benzotriazole-1-yl)-1,1,3,3-tetramethyluronium hexafluorophosphate (HBTU), 1-[bis(dimethylamino)methylene]-1H 1,2,3-triazolo[5 4,5-b]pyridinium 3-oxid hexafluorophosphate (HATU), 1-cyano-Z-ethoxy-2-oxoethylidenaminooxy)dimethylaminomorpholino-carbeniumhexafluorophosphate (COMU) and tetramethyl fluoroformamidinium hexafluorophosphate (TFFH) or mixtures thereof

The organic base is selected from Diisopropyl ethylamine (DIPEA), N-methylmorpholine (NMM), dimethylaminopyridine (DMAP), pyridine and the like. This is as shown in the scheme-3.

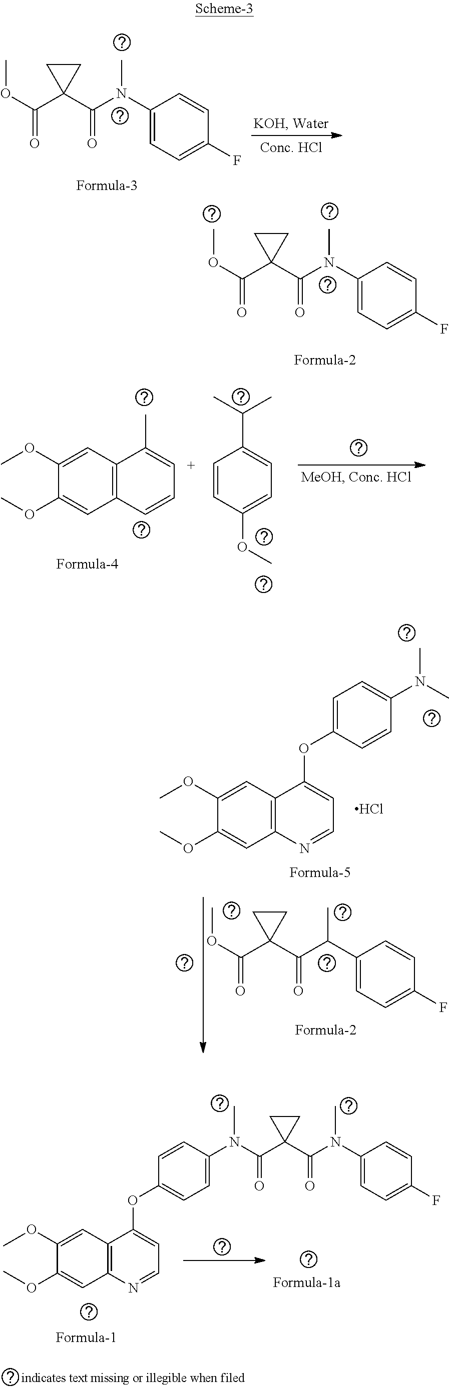

This scheme involves usage of coupling agents which are economically not viable. Coupling agents also lead to the formation of by-products/impurities. Coupling agent EDC·HCl, is one of the potential genotoxic agents.

This publication also discloses PyBOP, PyBrOP, COMU as additional coupling agents which are also expensive and lead to impurities.

Presently known methods have several disadvantages, including usage of coupling agents lead to the formation of by-products/impurities. Also, usage of PyBOP, PyBrOP, COMU as additional coupling agents which are expensive and lead to impurities. In addition, the benzotriazole motif has been reported to exhibit explosive properties, making scale-up and work at high temperatures difficult.

The prior art processes either use the acid chloride method or employ the condensation reagents for preparation of Cabozantinib via coupling of 1-(4-Fluoro-phenylcarbamoyl)-cyclopropanecarboxylic acid or its corresponding acid chloride with 4-(6,7-dimethoxy-quinoline-4-yloxy)-phenylamine.

### Disadvantages of Methods Involving Usage Acid Chloride:

This synthesis route to prepare Cabozantinib involves the formation of 1-(4-Fluoro-phenylcarbamoyl)-cyclopropanecarbonyl chloride by action of either thionyl chloride or oxalyl chloride on 1-(4-Fluoro-phenylcarbamoyl)-cyclopropanecarboxylic acid (Formula V) followed by condensation with 4-((6,7-dimethoxyquinolin-4-yl)oxy)aniline compound of (Formula III).

This route suffers from disadvantages like significant yield loss due to formation of impurities, hence not economically viable.

Disadvantages of usage of condensation reagents by acid-amine coupling method:

The usage of condensation reagents involves several disadvantages as below:
Reactions in general will not go for completion.
Reaction times are higher, resulting in higher cycle time and probability of impurity formation is higher.
The usage of condensation reagents renders the process costlier.
Formation of Impurity-1 (N¹,N²-bis(4-((6,7-dimethoxyquinolin-4-yl)oxy)phenyl)oxalamide) of structural formula IX.

It is therefore, needed to develop a process which not only overcomes the disadvantages of prior art but also is economical, operationally simple and industrially applicable.

The present invention provides an improved method for production of Cabozantinib by eliminating the steps of usage of acid chloride route or by usage of condensation agents.

### SUMMARY

Aspects of the present application provide a safe, simpler & economical process for the preparation of Cabozantinib. Each step of the process disclosed herein are contemplated both in the context of the multistep sequences described and individually.

One aspect of the invention discloses solid dispersion of Cabozantinib (S) Malate, and a pharmaceutically acceptable excipient.

Another aspect of the invention discloses a solid dispersion of Cabozantinib (S) Malate, and a pharmaceutically acceptable excipient in an amorphous form.

Another aspect of the invention discloses a solid dispersion, wherein the pharmaceutically acceptable excipient is selected from polyvinylpyrrolidone, cellulose derivative, polymethacrylate-based copolymers including methyl acrylate—methyl acrylic acid copolymers, colloidal silicon dioxide, polyhydric alcohol, polyethylene glycol, polyethylene oxide, polyoxyethylene derivative, polyvinyl alcohol, or propylene glycol derivative.

Another aspect of the invention discloses a process for preparing an amorphous Cabozantinib (S) Malate comprising:
a. dissolving Cabozantinib (S) Malate in a solvent,
b. removal of solvent, and
c. isolating amorphous Cabozantinib (S) Malate.

Another aspect of the invention discloses a process for preparing an amorphous Cabozantinib (S) Malate comprising:
a. dissolving Cabozantinib in a solvent,
b. Adding (S)-Malic acid and
c. isolating amorphous Cabozantinib (S) Malate by removal of solvent.

Another aspect of the invention discloses a process for preparing an amorphous Cabozantinib (S) Malate comprising:
a. dissolving Cabozantinib in a solvent,
b. adding (S)-Malic acid and at least one pharmaceutically acceptable excipient,
c. isolating amorphous Cabozantinib (S) Malate and a pharmaceutically acceptable excipient, by removal of solvent.

Another aspect of the invention discloses a process for preparing amorphous solid dispersion of Cabozantinib (S) Malate, and a pharmaceutically acceptable excipient comprising:
a. dissolving Cabozantinib (S) Malate in a solvent,
b. adding at least one pharmaceutically acceptable excipient,
c. removal of solvent, and
d. isolating amorphous solid dispersion of Cabozantinib (S) Malate, and a pharmaceutically acceptable excipient.

Another aspect of the invention discloses the process for preparation of solid dispersion comprising amorphous solid dispersion of Cabozantinib (S) Malate and a pharmaceutically acceptable excipient, wherein the solvent is selected from alcohol having 1-4 carbon atoms, alkyl nitrile having 1-4 carbon atoms, alkyl amide having 3-5 carbon atoms, aliphatic ether including cyclic ether having 1-4 carbon atoms, ketone having 3-9 carbon atoms, halogenated solvents having 1-4 carbon atoms or water or mixtures thereof.

Another aspect of the invention discloses the process for preparation of solid dispersion comprising amorphous solid dispersion of N-(4-(6,7-dimethoxyquinolin-4-yloxy)phenyl)-N′-(4-fluorophenyl) cyclopropane-1,1-dicarboxamide, (2S)-hydroxybutanedioate and a pharmaceutically acceptable excipient, wherein the solvent is selected from alcohol having 1-4 carbon atoms, alkyl nitrile having 1-4 carbon atoms, alkyl amide having 3-5 carbon atoms, aliphatic ether including cyclic ether having 1-4 carbon atoms, ketone having 3-9 carbon atoms, halogenated solvents having 1-4 carbon atoms or water or mixtures thereof.

One more aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating 1-(4-Fluoro-phenylcarbamoyl)-cyclopropanecarboxylic acid compound of Formula V using a suitable sulfonyl chloride, optionally in presence of a suitable base in a suitable solvent,
b) Adding compound of 4-((6,7-dimethoxyquinolin-4-yl)oxy)aniline compound of Formula III,
c) Isolating N-(4-(6,7-dimethoxyquinolin-4-yloxy)phenyl)-N′-(4-Fluorophenyl) cyclopropane-1,1-dicarboxamide compound of Formula II.

Wherein in step (a), suitable solvent is selected from halogenated solvents, ethereal solvents and nitrile solvents preferably, Dichloromethane, Dichloroethane, THF and acetonitrile.

Suitable sulfonyl chloride is selected from the list of Methane sulfonyl chloride (Mesyl chloride), p-Toluene sulfonyl chloride (Tosyl chloride), 4-Chlorobenzylsulfonyl chloride, 2-Chlorobenzylsulfonyl chloride, 4-Nitrophenyl sulfonyl chloride and the like.

Optionally, a suitable base is an organic base selected from N,N-Dimethylamino Pyridine (DMAP) and N-methylimidazole (NMI).

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating 1-(4-Fluoro-phenylcarbamoyl)-cyclopropanecarboxylic acid compound of Formula V using methane sulfonyl chloride, optionally in presence of a suitable base in dichloromethane,
b) Adding compound of 4-((6,7-dimethoxyquinolin-4-yl)oxy)aniline compound of Formula III,
a) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating 1-(4-Fluoro-phenylcarbamoyl)-cyclopropanecarboxylic acid compound of Formula V using methane sulfonyl chloride, optionally in presence of N,N-Dimethylamino Pyridine (DMAP) in dichloromethane,
b) Adding compound of 4-((6,7-dimethoxyquinolin-4-yl)oxy)aniline compound of Formula III
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating compound of 1-(4-Fluoro-phenylcarbamoyl)-cyclopropanecarboxylic acid Formula V using methane sulfonyl chloride, optionally in presence of N-methylimidazole (NMI) in dichloromethane,
b) Adding compound of 4-((6,7-dimethoxyquinolin-4-yl)oxy)aniline compound of Formula III
c) Isolating compound of N-(4-(6,7-dimethoxyquinolin-4-yloxy)phenyl)-N′-(4Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating compound of 1-(4-Fluoro-phenylcarbamoyl)-cyclopropanecarboxylic acid Formula V using p-Toluene sulfonyl chloride (Tosyl chloride), optionally in presence of a suitable base in dichloromethane,
b) Adding compound of 4-((6,7-dimethoxyquinolin-4-yl)oxy)aniline compound of Formula III
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating compound of 1-(4-Fluoro-phenylcarbamoyl)-cyclopropanecarboxylic acid Formula V using p-Toluene sulfonyl chloride (Tosyl chloride), optionally in presence of N,N-Dimethylamino Pyridine (DMAP) in dichloromethane,
b) Adding compound of 4-((6,7-dimethoxyquinolin-4-yl)oxy)aniline compound of Formula III,
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) Activating compound of 1-(4-Fluoro-phenylcarbamoyl)-cyclopropanecarboxylic acid Formula V using p-Toluene sulfonyl chloride (Tosyl chloride), optionally in presence of N-methylimidazole (NMI) in dichloromethane,
b) Adding compound of 4-((6,7-dimethoxyquinolin-4-yl)oxy)aniline compound of Formula III,
c) Isolating N-(4-(6,7-dimethoxyquinolin-4-yloxy)phenyl)-N′-(4-Fluorophenyl) cyclopropane-1,1-dicarboxamide compound of Formula II.

Yet another aspect of the invention discloses the preparation of N-(4-(6,7-dimethoxy quinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and compound of 4-((6,7-dimethoxyquinolin-4-yl)oxy)aniline compound of Formula III in a suitable solvent and a suitable base,
b) Adding a suitable sulfonyl chloride,
c) Isolating N-(4-(6,7-dimethoxyquinolin-4-yloxy)phenyl)-N′-(4-Fluorophenyl) cyclopropane-1,1-dicarboxamide compound of Formula II.

Wherein in step (a), suitable solvent is selected from halogenated solvents, ethereal solvents and nitrile solvents preferably, Dichloromethane, Dichloroethane, THF and acetonitrile.

Suitable sulfonyl chloride is selected from the list of Methane sulfonyl chloride (Mesyl chloride), p-Toluene sulfonyl chloride (Tosyl chloride), 4-Chlorobenzylsulfonyl chloride, 2-Chlorobenzylsulfonyl chloride, 4-Nitrophenyl sulfonyl chloride and the like.

Optionally, a suitable base is an organic base selected from N,N-Dimethylamino Pyridine (DMAP) and N-methylimidazole (NMI).

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and 4-((6,7-dimethoxyquinolin-4-yl)oxy)aniline compound of Formula III in dichloromethane and a suitable base,
b) Adding methane sulfonyl chloride,
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and 4-((6,7-dimethoxyquinolin-4-yl)oxy)aniline compound of Formula III in dichloromethane and N,N-Dimethylamino Pyridine (DMAP),
b) Adding methane sulfonyl chloride,
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and 4-((6,7-dimethoxyquinolin-4-yl)oxy)aniline compound of Formula III in dichloromethane and N-methylimidazole (NMI),
b) Adding methane sulfonyl chloride,
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and 4-((6,7-dimethoxyquinolin-4-yl)oxy)aniline compound of Formula III in dichloromethane and a suitable base,
b) Adding p-Toluene sulfonyl chloride (Tosyl chloride),
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and 4-((6,7-dimethoxyquinolin-4-yl)oxy)aniline compound of Formula III in dichloromethane and N,N-Dimethylamino Pyridine (DMAP),
b) Adding p-Toluene sulfonyl chloride (Tosyl chloride),
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the invention discloses the preparation of N-(4-(6,7-dimethoxyquinolin-4-yloxy)phenyl)-N′-(4Fluorophenyl) cyclopropane-1,1-dicarboxamide compound of Formula II comprising of following steps.
a) To a solution of compound of 1-(4-Fluoro-phenylcarbamoyl)-cyclopropanecarboxylic acid Formula V and 4-((6,7-dimethoxyquinolin-4-yl)oxy)aniline compound of Formula III in dichloromethane and N-methylimidazole (NMI),
b) Adding p-Toluene sulfonyl chloride (Tosyl chloride),
c) Isolating N-(4-(6,7-dimethoxyquinolin-4-yloxy)phenyl)-N′-(4-Fluorophenyl) cyclopropane-1,1-dicarboxamide compound of Formula II.

Another aspect of the present invention is to provide a novel crystalline form of N-(4-(6,7-dimethoxyquinolin-4-yloxy)phenyl)-N′-(4Fluorophenyl) cyclopropane-1,1-dicarboxamide compound of Formula II characterized by an X-ray diffraction including peaks at 8.90, 10.60, 13.20, 13.70, and 21.80 degrees 2θ, +/−0.2 degrees, using Cu-Kα radiation.

Another aspect of the present invention discloses a process for the preparation of amorphous Cabozantinib (S) Malate of Formula I comprising of following steps.
a) Heating Cabozantinib of Formula (II) in a first solvent mixture to 40-45° C. and filtered,
b) Filterate was concentrated and diluted with a second solvent mixture and filtered,
c) Filterate was concentrated to obtain amorphous Cabozantinib (S) Malate of Formula I.
Wherein the first solvent mixture comprises a mixture of ether solvent and alcoholic solvents and second solvent mixture comprises of chlorinated solvents & alcoholic solvents.
The ether solvents selected from Tetrahydrofuran, tetrahydropyran, t-Butyl ether, Methyl t-Butyl ether, n-butyl ether and the like; chlorinated solvents selected from dichloromethane, dichloroethane, chloroform, carbon tetrachloride and the like; alcohol solvents selected from methanol, ethanol, propanol and the like.

Another aspect of the invention discloses Formula XI (Impurity-3). Formula XI (Impurity-3) is a ring opened impurity due to the reaction between Formula II and HCl (a by-product).

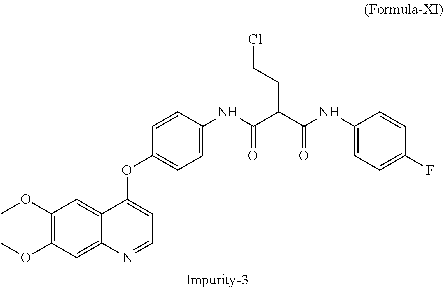 [SMILES: COc1cc2nccc(Oc3ccc(NC(=O)C(CCCl)C(=O)Nc4ccc(F)cc4)cc3)c2cc1OC]

Another aspect of the invention discloses Formula X (Impurity-2) (N-(4-((6,7-dimethoxy-quinolin-4-yl)oxy)phenyl)-methanesulfonamide) of structural formula X and the reasoning for the formation thereof:

Formula X (Impurity 2) is a mesylated of impurity 4-((6,7-dimethoxyquinolin-4-yl)oxy)aniline compound of Formula III impurity due to the reaction between 4-((6,7-dimethoxyquinolin-4-yl)oxy)aniline compound of Formula III and Mesyl chloride.

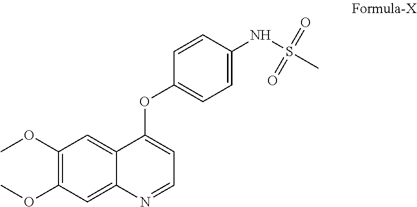

It was found that the Impurity-2 (N-(4-((6,7-dimethoxy-quinolin-4-yl)oxy)phenyl)-methanesulfonamide) of Formula X was a resultant of the competitive side reaction of methane sulfonyl chloride and 4-((6,7-dimethoxyquinolin-4-yl)oxy)aniline compound of Formula III.

The present invention has also been designed to control the competitive side reaction to form Impurity-2 (N-(4-((6,7-dimethoxy-quinolin-4-yl)oxy)phenyl)-methanesulfonamide) of Formula X by using the optimised molar equivalents of the reagents.

Another aspect of the invention discloses Formula XIV (Impurity-6) (1,3-bis(4-((6,7-dimethoxyquinolin-4-yl)oxy)phenyl)urea).

The present invention has also been designed to control the competitive side reaction to form Impurity-6 (Impurity-6) (1,3-bis(4-((6,7-dimethoxyquinolin-4-yl)oxy)phenyl)urea) of Formula XIV by using the optimised molar equivalents of the reagents.

### BRIEF DESCRIPTION OF DRAWINGS

FIG. 1: Illustrates the XRD pattern of crystalline N-(4-(6,7-dimethoxyquinolin-4-yloxy)phenyl)-N′-(4fluorophenyl)cyclopropane-1,1-dicarboxamide, prepared according to Example 11.

FIG. 2: Illustrates the XRD pattern of amorphous solid dispersion of N-(4-(6,7-dimethoxyquinolin-4-yloxy)phenyl)-N′-(4fluorophenyl)cyclopropane-1,1-dicarboxamide, (2S)-hydroxybutanedioate, and Eudragit, prepared according to Example 24.

### SYNTHETIC SCHEME OF THE PRESENT INVENTION

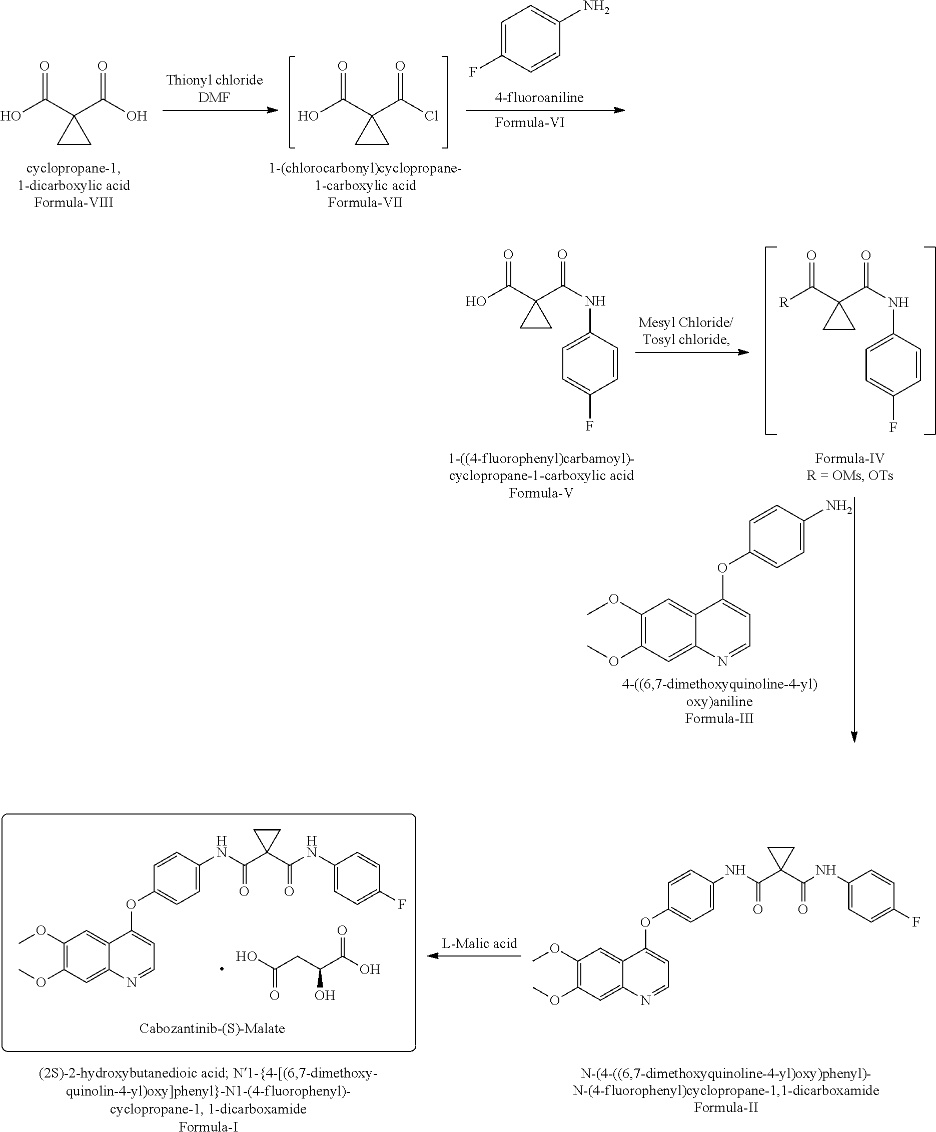

The present invention describes the synthesis of Cabozantinib, by the mixed anhydride method using different counter acids. This method is recognized as a rational process due to its simplicity, commercial availability of acid chlorides and atom economy. The present invention involves the usage of methane sulfonyl chloride or toluene sulfonyl chloride. Methane sulfonyl chloride is used for activating 1-(4-Fluoro-phenylcarbamoyl)-cyclopropanecarboxylic acid (Formula V) and the reaction proceeds via mixed anhydride method.

The residual methane sulfonyl chloride or toluene sulfonyl chloride is quenched with water and removed as a salt of N,N-Dimethylpyridin-4-amine (DMAP) or 1-Methylimidazole (NMI) in aqueous layer.

### Mixed Anhydride Route of Synthesis of Cabozantinib of Present Invention

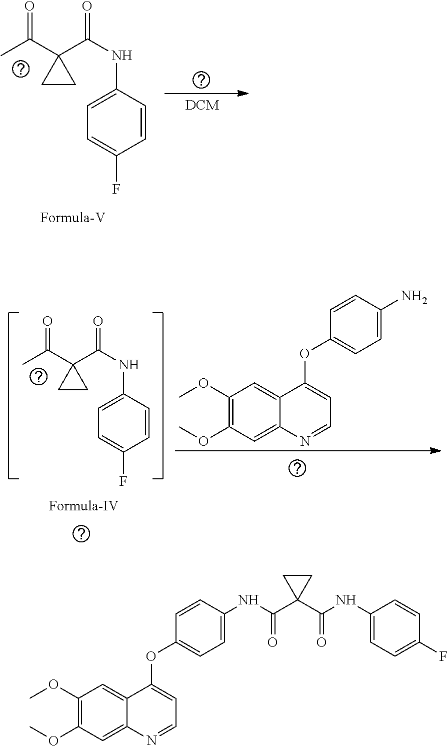

The synthesis of Cabozantinib of present invention involves the usage of mixed anhydride method. The mechanism of the reaction is illustrated as below.

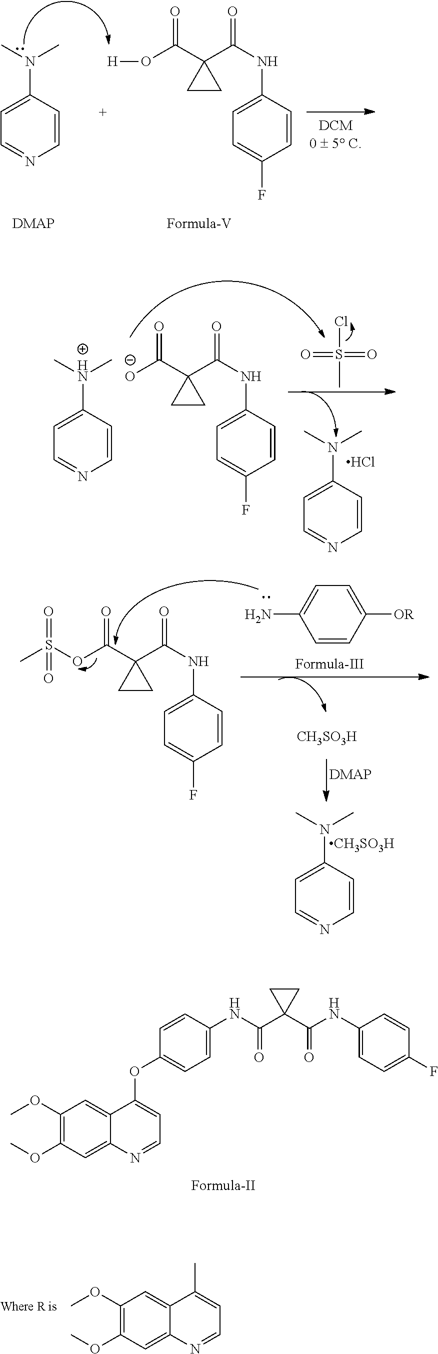

### Advantages of the Present Invention

1-(4-Fluoro-phenylcarbamoyl)-cyclopropanecarboxylic acid (Formula V) is isolated without the assistance of base.
Cyclopropane-1,1-dicarboxylic acid (Formula VIII—a potential impurity for the subsequent step) is purged during the workup/isolation.
The present process for the synthesis of Cabozantinib, by the mixed anhydride method using different counter acids. This method is recognized as a rational process due to its simplicity, commercial availability of acid chlorides and atom economy. The present invention involves the usage of methane sulfonyl chloride or toluene sulfonyl chloride. Methane sulfonyl chloride is used for activating 1-(4-Fluoro-phenylcarbamoyl)-cyclopropanecarboxylic acid (Formula V).
N-Methyl Imidazole is commercially available and economic. Reactions using this reagent can be handled on large scale. Being a liquid, handling is relatively better considering the purge of residual compound. N-Methyl imidazole is being removed during the aqueous work up and hence does not affect on the quality of N-(4-(6,7-dimethoxyquinolin-4-yloxy)phenyl)-N′-(4Fluorophenyl) cyclopropane-1,1-dicarboxamide compound of Formula II.
This process is both cost-effective and suitable for large-scale production with high yield.

### Impurities Associated with Synthesis of N-(4-(6,7-dimethoxyquinolin-4-yloxy)phenyl)-N′-(4Fluorophenyl) cyclopropane-1,1-dicarboxamide Compound of Formula II (Cabozantinib)

One of the key advantages of this invention is that the process developed for the preparation of N-(4-(6,7-dimethoxyquinolin-4-yloxy)phenyl)-N′-(4-fluorophenyl)cyclopropane-1,1-dicarboxamide, (2S)-hydroxy butanedioate compound of Formula I is capable of controlling the following impurities with a limit of NMT 0.15%.

Following is the list of impurities identified during the synthesis of N-(4-(6,7-dimethoxyquinolin-4-yloxy)phenyl)-N′-(4Fluorophenyl) cyclopropane-1,1-dicarboxamide compound of Formula II (Cabozantinib). These impurities are caused due to various synthetic routes used for the preparation of N-(4-(6,7-dimethoxyquinolin-4-yloxy)phenyl)-N′-(4Fluorophenyl) cyclopropane-1,1-dicarboxamide compound of Formula II (Cabozantinib).

| Name of the impurity | Chemical Name | Structure |
|---|---|---|
| Impurity-1 | N¹,N²-bis(4-((6,7- dimethoxyquinolin-4- yl)oxy)phenyl)oxalamide | 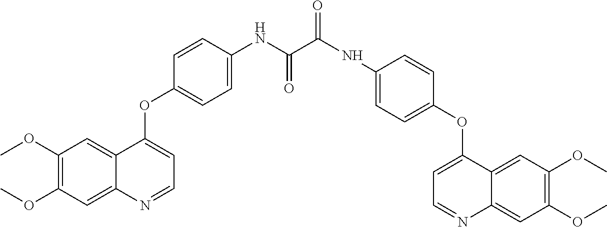 |
| | | Formula IX |
| Impurity-2 | N-(4-((6,7-dimethoxy- quinolin-4- yl)oxy)phenyl)- methanesulfonamide | 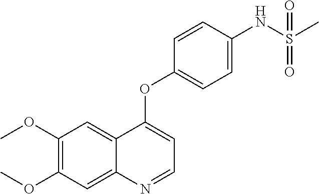 |
| | | Formula X |
| Impurity-3 | 2-(2-chloroethyl)-N-(4- ((6,7-dimethoxyquinolin- 4-yl)oxy)phenyl)-N′-3- (4- fluorophenyl) malonamide | 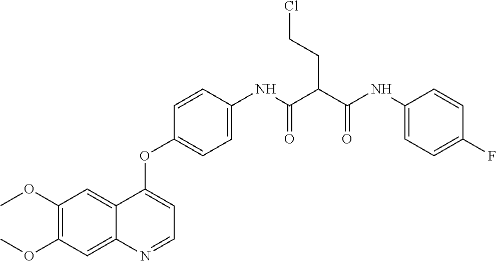 [SMILES: COc1cc2nccc(Oc3ccc(NC(=O)C(CCCl)C(=O)Nc4ccc(F)cc4)cc3)c2cc1OC] |
| | | Formula XI |
| Impurity-4 | N,N′-bis(4-fluorophenyl)- cyclopropane-1,1- dicarboxamide | 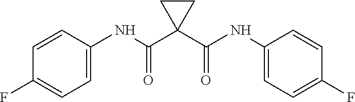 [SMILES: O=C(Nc1ccc(F)cc1)C1(C(=O)Nc2ccc(F)cc2)CC1] |
| | | Formula XII |
| Impurity-5 | N,N′-bis(4-((6,7- dimethoxyquinolin-4- yl)oxy)phenyl)cyclopro- pane-1,1-dicarboxamide | 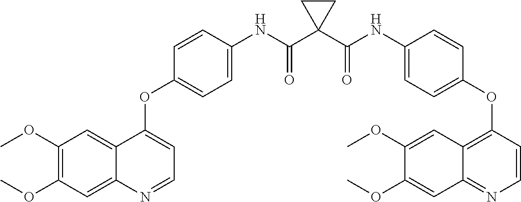 [SMILES: COc1cc2nccc(Oc3ccc(NC(=O)C4(C(=O)Nc5ccc(Oc6ccnc7cc(OC)c(OC)cc67)cc5)CC4)cc3)c2cc1OC] |
| | | Formula XIII |
| Impurity-6 | 1,3-bis(4-((6,7- dimethoxyquinolin-4- yl)oxy)phenyl)urea | 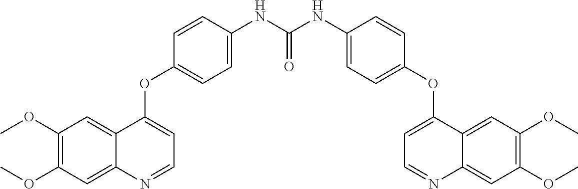 |
| | | Formula XIV |

The causes of the formation of impurities and the steps taken to mitigate or to minimize the formation of these impurities are also disclosed below:

Salient feature of this invention is that Cyclopropane-1,1-dicarboxylic acid compound of Formula VIII is controlled by design which facilitates the control of Impurity-4 (N,N′-bis(4-fluorophenyl)-cyclopropane-1,1-dicarboxamide) of Formula XII and Impurity 5 (which are due the side reaction between Cyclopropane-1,1-dicarboxylic acid compound of Formula VIII and 4-fluoroaniline compound of Formula VI and 4-((6,7-dimethoxyquinolin-4-yl)oxy)aniline compound of Formula III respectively).

Impurity-1 (N¹,N²-bis(4-((6,7-dimethoxyquinolin-4-yl)oxy)phenyl)oxalamide) of Structural Formula IX and the Reasoning for the Formation Thereof:

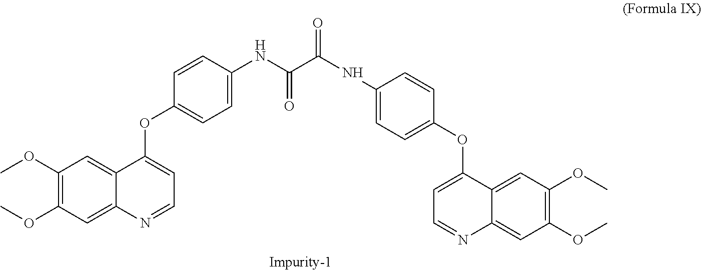

One of the possible routes of formation of this impurity is due to the reaction between oxalyl chloride (mentioned in most of the processes in the literature) and 4-((6,7-dimethoxyquinolin-4-yl)oxy)aniline compound of Formula III.

Formula XI (Impurity-3) is a ring opened impurity due to the reaction between Formula II and HCl (a by-product).

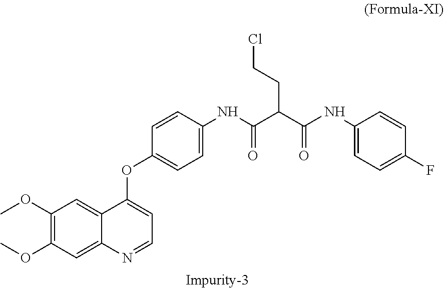 [SMILES: COc1cc2nccc(Oc3ccc(NC(=O)C(CCCl)C(=O)Nc4ccc(F)cc4)cc3)c2cc1OC]

Impurity-2 (N-(4-((6,7-dimethoxy-quinolin-4-yl)oxy)phenyl)-methanesulfonamide) of Structural Formula X and the Reasoning for the Formation Thereof:

Formula X (Impurity 2) is a mesylated of impurity 4-((6,7-dimethoxyquinolin-4-yl)oxy)aniline compound of Formula III impurity due to the reaction between 4-((6,7-dimethoxyquinolin-4-yl)oxy)aniline compound of Formula III and Mesyl chloride.

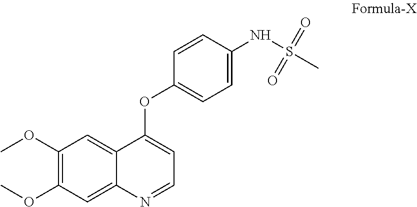

Another important aspect of the present invention is the identification, isolation and characterization of the Impurity-2 (N-(4-((6,7-dimethoxy-quinolin-4-yl)oxy)phenyl)-methanesulfonamide) of Formula X. It was found that the Impurity-2 (N-(4-((6,7-dimethoxy-quinolin-4-yl)oxy)phenyl)-methanesulfonamide) of Formula X was a resultant of the competitive side reaction of methane sulfonyl chloride and 4-((6,7-dimethoxyquinolin-4-yl)oxy)aniline compound of Formula III.

The present invention has also been designed to control the competitive side reaction to form Impurity-2 (N-(4-((6,7-dimethoxy-quinolin-4-yl)oxy)phenyl)-methanesulfonamide) of Formula X by using the optimised molar equivalents of the reagents.

Impurity-4 (N,N′-bis(4-fluorophenyl)-cyclopropane-1,1-dicarboxamide) of Formula XII is due to the reaction between 4-fluoroaniline compound of Formula VI and 1-(4-Fluoro-phenylcarbamoyl)-cyclopropanecarboxylic acid compound of Formula V.

Impurity-5 (N,N′-bis(4-((6,7-dimethoxy quinolin-4-yl)oxy)phenyl)cyclopropane-1,1-dicarboxamide) of Formula XIII is due to the reaction between 4-((6,7-dimethoxyquinolin-4-yl)oxy)aniline compound of Formula III and Cyclopropane-1,1-dicarboxylic acid compound of Formula VIII.

The Present Invention Also Provides for Amorphous Dispersion of N-(4-(6,7-dimethoxyquinolin-4-yloxy) phenyl)-N′-(4-fluorophenyl) cyclopropane-1,1-dicarboxamide with Pharmaceutically Acceptable Excipient:

Amorphous dispersions have been prepared by various techniques like concentration of solvents under vacuum, vacuum tray drying and spray drying. In a general procedure, a solution of Formula I (prepared by contacting Formula II with L-Malic acid) along with suitable pharmaceutically acceptable excipient is subjected for the removal of volatiles under reduced pressure to obtain Amorphous dispersion of Cabozantinib-S-Malate. Pharmaceutically acceptable excipient of different ratios has been studied for the preparation of Amorphous dispersions.

In order to understand the long term stability under forced conditions, thermal stress studies have been performed for the Amorphous dispersions. Those with polymethacrylate-based copolymers including methyl acrylate—methyl acrylic acid copolymers such as Eudragit have been found to be stable when stored 25° C.±2° C. and 40° C.±2° C.

Performance of Amorphous dispersion with Eudragit was found to be comparable with that the Innovator form (N2) in terms of solubility.

### Advantages of Eudragit:

Present as Powder, easy to handle.
Thermally stable.
Glass transition temperature greater than 150° C.
Will assist for attaining long term stability.
Pharmaceutically acceptable Inactive Ingredient.

### Characterization Methods:

PXRD was used for characterising amorphous N-(4-(6,7-dimethoxyquinolin-4-yloxy)phenyl)-N′-(4-fluorophenyl) cyclopropane-1,1-dicarboxamide, (2S)-hydroxybutanedioate and amorphous solid dispersion of N-(4-(6,7-dimethoxyquinolin-4-yloxy)phenyl)-N′-(4-fluorophenyl) cyclopropane-1,1-dicarboxamide, (2S)-hydroxybutanedioate and a pharmaceutically acceptable excipient. The Powder X-ray diffraction is one of the most used techniques to determine different crystalline and amorphous structures.

### General Procedure:

General Procedure—1: Preparation of Amorphous N-(4-(6,7-dimethoxyquinolin-4-yloxy)phenyl)-N′-(4-fluorophenyl) cyclopropane-1,1-dicarboxamide, (2S)-hydroxybutanedioate.

To a glass vessel equipped with a stirrer, condenser and a thermometer probe were added N-(4-(6,7-dimethoxyquinolin-4-yloxy)phenyl)-N′-(4-fluorophenyl) cyclopropane-1,1-dicarboxamide, (2S)-hydroxybutanedioate solution. The reaction mixture was stirred. The solvent was removed to isolate Amorphous N-(4-(6,7-dimethoxyquinolin-4-yloxy)phenyl)-N′-(4-fluorophenyl) cyclopropane-1,1-dicarboxamide, (2S)-hydroxybutanedioate.

Solvent is selected from alcohol having 1-4 carbon atoms, alkyl nitrile having 1-4 carbon atoms, alkyl amide having 3-5 carbon atoms, aliphatic ether including cyclic ether having 1-4 carbon atoms, ketone having 3-9 carbon atoms, halogenated solvents having 1-4 carbon atoms or water or mixtures thereof.

General Procedure—2: Preparation of Amorphous N-(4-(6,7-dimethoxyquinolin-4-yloxy)phenyl)-N′-(4-fluorophenyl) cyclopropane-1,1-dicarboxamide, (2S)-hydroxybutanedioate.

To a glass vessel equipped with a stirrer, condenser and a thermometer probe were added N-(4-(6,7-dimethoxyquinolin-4-yloxy)phenyl)-N′-(4-fluorophenyl) cyclopropane-1,1-dicarboxamide solution. (2S)-2-Hydroxybutanedioic acid was added to the above solution and the reaction mixture was stirred. The solvent was removed to isolate Amorphous N-(4-(6,7-dimethoxyquinolin-4-yloxy)phenyl)-N′-(4-fluorophenyl) cyclopropane-1,1-dicarboxamide, (2S)-hydroxybutanedioate.

Solvent is selected from alcohol having 1-4 carbon atoms, alkyl nitrile having 1-4 carbon atoms, alkyl amide having 3-5 carbon atoms, aliphatic ether including cyclic ether having 1-4 carbon atoms, ketone having 3-9 carbon atoms, halogenated solvents having 1-4 carbon atoms or water or mixtures thereof.

General Procedure—3: Preparation of Amorphous Solid Dispersion of N-(4-(6,7-dimethoxyquinolin-4-yloxy)phenyl)-N′-(4-fluorophenyl) cyclopropane-1,1-dicarboxamide, (2S)-hydroxybutanedioate and a Pharmaceutically Acceptable Excipient.

To a glass vessel equipped with a stirrer, condenser and a thermometer probe were added N-(4-(6,7-dimethoxyquinolin-4-yloxy)phenyl)-N′-(4-fluorophenyl) cyclopropane-1,1-dicarboxamide, (2S)-hydroxybutanedioate solution and a pharmaceutically acceptable excipient. The reaction mixture was stirred. The solvents were removed to isolate amorphous solid dispersion of N-(4-(6,7-dimethoxyquinolin-4-yloxy)phenyl)-N′-(4-fluorophenyl) cyclopropane-1,1-dicarboxamide, (2S)-hydroxybutanedioate with a pharmaceutically acceptable excipient.

Solvent is selected from alcohol having 1-4 carbon atoms, alkyl nitrile having 1-4 carbon atoms, alkyl amide having 3-5 carbon atoms, aliphatic ether including cyclic ether having 1-4 carbon atoms, ketone having 3-9 carbon atoms, halogenated solvents having 1-4 carbon atoms or water or mixtures thereof.

General Procedure—4: Preparation of Amorphous Solid Dispersion of N-(4-(6,7-dimethoxyquinolin-4-yloxy)phenyl)-N′-(4-fluorophenyl) cyclopropane-1,1-dicarboxamide, (2S)-hydroxybutanedioate and a Pharmaceutically Acceptable Excipient.

To a glass vessel equipped with a stirrer, condenser and a thermometer probe were added N-(4-(6,7-dimethoxyquinolin-4-yloxy)phenyl)-N′-(4-fluorophenyl) cyclopropane-1,1-dicarboxamide, (2S)-hydroxybutanedioate solution. Adding (2S)-2-Hydroxybutanedioic acid and a pharmaceutically acceptable excipient. The reaction mixture was stirred. The solvents were removed to isolate amorphous solid dispersion of N-(4-(6,7-dimethoxyquinolin-4-yloxy)phenyl)-N′-(4-fluorophenyl) cyclopropane-1,1-dicarboxamide, (2S)-hydroxybutanedioate with a pharmaceutically acceptable excipient.

Solvent is selected from alcohol having 1-4 carbon atoms, alkyl nitrile having 1-4 carbon atoms, alkyl amide having 3-5 carbon atoms, aliphatic ether including cyclic ether having 1-4 carbon atoms, ketone having 3-9 carbon atoms, halogenated solvents having 1-4 carbon atoms or water or mixtures thereof.

The input N-(4-(6,7-dimethoxyquinolin-4-yloxy)phenyl)-N′-(4-fluorophenyl) cyclopropane-1,1-dicarboxamide, (2S)-hydroxybutanedioate for formula (I) as disclosed in above mentioned general procedures could be either in crystalline form or amorphous form.

The scheme is represented by following examples. These examples are for illustration only and hence should not be construed as limitation of the scope of the invention.

### Example-1: Preparation of 1-(4-Fluoro-phenylcarbamoyl)-cyclopropanecarboxylic acid (Formula V)

To a glass vessel equipped with a stirrer, condenser, a thermometer probe and under nitrogen atmosphere were added the Cyclopropane-1,1-dicarboxylic acid compound of Formula VIII (50.0 g, 1.00 equiv.) and 2-Methyl THF (500 mL, 10.0 vol.). The mass was cooled to 0-5° C. and thionyl chloride (1.20 equiv.) was added dropwise. The reaction mass stirred for 1 h. In the meanwhile, 4-fluoroaniline compound of Formula VI (42.70 g, 1.00 equiv.) was diluted with 2-Methyl THF (100 mL, 2.00 vol.). Diluted solution of 4-fluoroaniline compound of Formula VI was added to the reaction mass maintaining 0-5° C. The reaction mass was further stirred for 2 h maintaining the same temperature. Water (250 mL, 5.00 vol.) was added to the reaction mass at 0-5° C. followed by acid base work up. The mass was filtered, washed with water and dried at 40-50° C. under vacuum to obtain 1-(4-Fluoro-phenylcarbamoyl)- cyclopropanecarboxylic acid compound of Formula V as a solid (45.0 g).

### Example-2: Preparation of 1-(4-Fluoro-phenylcarbamoyl)-cyclopropanecarboxylic acid (Formula V)

To a glass vessel equipped with a stirrer, condenser, a thermometer probe and under nitrogen atmosphere were added the Cyclopropane-1,1-dicarboxylic acid compound of Formula VIII (25.0 g, 1.00 equiv.) and 2-Methyl THF (250 mL, 10.0 vol.). The mass was cooled to 0-5° C. and thionyl chloride (1.20 equiv.) was added dropwise. The reaction mass stirred for 1 h.

In another glass vessel equipped with a stirrer, condenser, a thermometer probe and under nitrogen atmosphere were added the 4-fluoroaniline compound of Formula VI (21.35 g, 1.00 equiv.) and 2-Methyl THF (50 mL, 2.00 vol.). The mass was cooled under nitrogen and 1-(chlorocarbonyl) cyclopropane-1-carboxylic acid compound of Formula VII solution (prepared as above) was added maintaining temperature of 0-5° C. The reaction mass maintained for 2 h. Water (125 mL, 5.00 vol.) was added followed acid base work up resulted in 1-(4-Fluoro-phenylcarbamoyl)-cyclopropanecarboxylic acid compound of Formula V (27.30 g)

### Example-3: Preparation of 1-(4-Fluoro-phenylcarbamoyl)-cyclopropanecarboxylic acid (Formula V)

To a glass vessel equipped with a stirrer, condenser, a thermometer probe and under nitrogen atmosphere were added the Cyclopropane-1,1-dicarboxylic acid compound of Formula VIII (50.0 g, 1.00 equiv.), 2-Methyl THF (100 mL, 2.00 vol.), cooled to 0-5° C., triethylamine (42.77 g, 1.10 equiv.) dropwise added, thionyl chloride (54.83 g, 1.20 equiv.) dropwise added, the reaction mass was stirred for 1 h, diluted 4-fluoroaniline compound of Formula VI (42.70 g, 1.00 equiv.) in 2-Methyl THF (250 mL, 5.00 vol.) was added at 0-5° C., the reaction mass maintained for 2 h. Water (250 mL, 5.00 vol.) was added followed acid base work up resulted in 1-(4-Fluoro-phenylcarbamoyl)-cyclopropanecarboxylic acid compound of Formula V (45.5 g).

### Example-4: Preparation of 1-(4-Fluoro-phenylcarbamoyl)-cyclopropanecarboxylic acid (Formula V)

To a glass vessel equipped with a stirrer, condenser, a thermometer probe and under nitrogen atmosphere were added the Cyclopropane-1,1-dicarboxylic acid compound of Formula VIII (50.0 g, 1.00 equiv.), 2-Methyl THF (100 mL, 2.00 vol.), cooled to 0-5° C., triethylamine (42.77 g, 1.10 equiv.) dropwise added, thionyl chloride (54.83 g, 1.20 equiv.) dropwise added, the reaction mass was stirred for 1 h. The mass was filtered and the solid was washed with 2-MeTHF (250 mL, 5.0 vol.). This constitutes Formula VII solution.

In another glass vessel equipped with a stirrer, condenser, a thermometer probe and under nitrogen atmosphere were added the 4-fluoroaniline compound of Formula VI (44.80 g, 1.05 equiv.) and 2-Methyl THF (100.0 mL, 2.00 vol.). The mass was cooled under nitrogen and 1-(chlorocarbonyl)cyclopropane-1-carboxylic acid compound of Formula VII solution (prepared as above) was added maintaining temperature of 0-5° C. The reaction mass maintained for 2 h. Water (250 mL, 5.00 vol.) was added followed acid base work up resulted in 1-(4-Fluoro-phenylcarbamoyl)-cyclopropanecarboxylic acid compound of Formula V (54.0 g).

### Example-5: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermoprobe and heating cooling system were added 1-(4-Fluoro-phenylcarbamoyl)-cyclopropanecarboxylic acid compound of Formula V (50.0 g,1.0 equiv) and Dichloromethane (1.0 L, 20.0 vol) under nitrogen atmosphere. DMAP (68.48 g, 2.5 equiv) was added and the mixture was cooled between 0 and 5° C. Methane sulfonyl chloride (25.68 g, 1.0 equiv) was added maintaining temperature between 0 and 5° C. The mass was stirred at 0-5° C. under nitrogen atmosphere. 4-((6,7-dimethoxyquinolin-4-yl)oxy)aniline compound of Formula III (56.47 g) was added and mass was stirred at 0-5° C. After completion of reaction, water (50.0 mL, 1.0 vol), was added followed by Methanol (100 mL, 2.0 vol) and layers were separated. Organic layer was concentrated under reduced pressure, methanol (750 mL, 15.0 vol) was added and mass was stirred at 55-60° C., cooled to 25-30° C., filtered, washed with methanol (150 mL, 3.0 vol) and dried the solid at 40 to 50° C. under vacuum to get N-(4-(6,7-dimethoxyquinolin-4-yloxy)phenyl)-N′-(4Fluorophenyl)cyclopropane-1,1-dicarboxamide compound of Formula II as a solid. Formula II was characterised by XRD and HPLC.

### Example-6: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermoprobe and heating cooling system were added 1-(4-Fluoro-phenylcarbamoyl)-cyclopropanecarboxylic acid compound of Formula V (50.0 g, 1.0 equiv) and Dichloromethane (1.0 L, 20.0 vol) under nitrogen atmosphere. DMAP (68.48 g, 2.5 equiv) was added and the mixture was cooled between 0 and5° C. Methane sulfonyl chloride (25.68 g, 1.0 equiv) was added maintaining temperature between 0 and 5° C. The mass was stirred at 0-5° C. under nitrogen atmosphere. 4-((6,7-dimethoxyquinolin-4-yl)oxy)aniline compound of Formula III (59.79 g was added and mass was stirred at 0-5° C. After completion of reaction, water (50.0 ml, 1.0 vol), was added followed by MeOH (100 mL, 2.0 vol). Layers were separated. Organic layer was concentrated the under reduced pressure, Acetonitrile (750 mL, 15.0 vol) and water (50.0 mL, 1.0 vol) were added. Mass was heated to 55-60° C., cooled to 25-30° C., filtered, washed with acetonitrile (150 mL, 3.0 vol) and dried the solid at 50-55° C. under vacuum to get Formula II as a solid. The solid obtained was characterized by XRD and HPLC.

### Example-7: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermoprobe and heating cooling system were added 1-(4-Fluoro-phenylcarbamoyl)-cyclopropanecarboxylic acid compound of Formula V (5.00 g,1.0 equiv) and Dichloromethane (100 mL, 20.0 vol). 1-Methyl imidazole (5.42 g, 3.0 equiv) was added and the reaction mass was cooled to 0-5° C. Methane sulfonyl chloride (2.568 g, 1.0 equiv) was added maintaining temperature at 0-5° C. under nitrogen atmosphere. 4-((6,7-dimethoxyquinolin-4-yl)oxy)aniline compound of Formula III (5.647 g, 0.85 equiv) was added. After completion of reaction, water (5.0 ml, 1.0 vol) was added followed by MeOH (10.0 mL, 2.0 vol) and layers were separated. Organic layer was concentrated under reduced pressure, methanol (75.0 mL,15.0 vol) was added and mass was stirred at 55-60° C. It was cooled to 25-30° C., filtered, washed with methanol (15.0 ml, 3.0 vol) and dried the solid at 50-55° C. under vacuum to get N-(4-(6,7-dimethoxyquinolin-4-yloxy)phenyl)-N′-(4-Fluorophenyl) cyclopropane-1,1-dicarboxamide compound of Formula II as a solid. The solid obtained was characterized by XRD and HPLC.

### Example-8: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermoprobe and heating cooling system were added 1-(4-Fluoro-phenylcarbamoyl)-cyclopropanecarboxylic acid compound of Formula V (2.00 g, 1.0 equiv) and Dichloromethane (40 mL, 20.0 vol). DMAP (3.28, 3.0 equiv) was added and the reaction mass was cooled to 0-5° C. Tosyl chloride (2.568 g, 1.0 equiv) was added maintaining temperature at 0-5° C. under nitrogen atmosphere. 4-((6,7-dimethoxyquinolin-4-yl)oxy)aniline compound of Formula III (2.22 g, 0.85 equiv) was added, raised the reaction mass temperature to 25-30° C. and stirred for 120 min at 25-30° C. After completion of reaction, water (10.0 ml, 5.0 vol) was added and layers were separated. Organic layer was concentrated under reduced pressure, methanol (30.0 mL, 15.0 vol) was added and mass was stirred at 60-65° C. It was cooled to 25-30° C., filtered, washed with methanol (15.0 mL, 3.0 vol) and dried the solid at 50-55° C. under vacuum to get N-(4-(6,7-dimethoxyquinolin-4-yloxy)phenyl)-N′-(4-Fluorophenyl) cyclopropane-1,1-dicarboxamide compound of Formula II as a solid.

### Example-9: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermometer sensor and heating cooling system were added 1-(4-Fluoro-phenylcarbamoyl)-cyclopropanecarboxylic acid compound of Formula V (25.0 g, 1.0 equiv.), 4-((6,7-dimethoxyquinolin-4-yl)oxy)aniline compound of Formula III (28.2 g, 0.85 equiv.), DMAP (34.2 g, 2.5 equiv.) and Dichloromethane (0.5 L, 20.0 vol) under nitrogen atmosphere and the mixture was cooled to 0-5° C. Methane sulfonyl chloride (16.16 g,1.26 equiv.) was added by maintaining the temperature between 0 and 5° C. The mass was stirred at 10-15° C. for 2 h. After completion of the reaction, water (50.0 mL, 2.0 vol) was added and allowed to 25-30° C. and layers were separated. Organic layer was concentrated under reduced pressure, methanol (625 mL, 25.0 vol) was added and mass was stirred at 55-60° C., cooled to 25-30° C., filtered, washed with methanol (125 mL, 5.00 vol) and dried the solid at 45 to 50° C. under vacuum to obtain N-(4-(6,7-dimethoxyquinolin-4-yloxy)phenyl)-N′-(4-Fluorophenyl) cyclopropane-1,1-dicarboxamide compound of Formula II.

### Example-10: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermoprobe and heating cooling system were added 1-(4-Fluoro-phenylcarbamoyl)-cyclopropanecarboxylic acid compound of Formula V (4.00 g, 1.0 equiv) and Dichloromethane (100 mL, 25.0 vol). CDI (2.90 g, 1.0 equiv), Imidazole (1.21g, 1.0 equiv) was added, temperature of the reaction mass was raised to 35-40° C. and stirred the reaction for 2 h at 35-40° C. under nitrogen atmosphere. 4-((6,7-dimethoxyquinolin-4-yl)oxy)aniline compound of Formula III (4.5 g, 0.85 equiv) was added. After completion of reaction, cooled the reaction to 25-30° C., 20% Sodium carbonate solution (40.0 ml, 10.0 vol) was added and layers were separated. Washed the organic layer with brine solution (40.0 ml, 10.0 vol), Organic layer was concentrated under reduced pressure, methanol (75.0 mL, 15.0 vol) was added and mass was stirred at 55-60° C. It was cooled to 25-30° C., filtered, washed with methanol (20.0 ml, 5.0 vol) and dried the solid at 65° C. under vacuum to get N-(4-(6,7-dimethoxyquinolin-4-yloxy)phenyl)-N′-(4Fluorophenyl) cyclopropane-1,1-dicarboxamide compound of Formula II as a solid.

### Example-11: Preparation of N-(4-(6,7-dimethoxyquinolin-4-yloxy)phenyl)-N′-(4 Fluorophenyl) cyclopropane-1,1-dicarboxamide (Formula II)

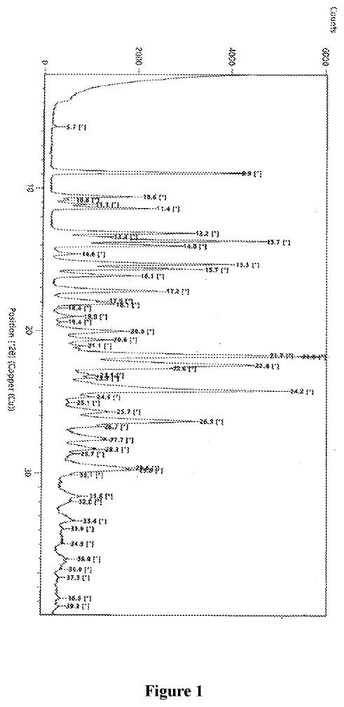

To a reactor equipped with a stirrer, thermometer sensor and heating cooling system were added 1-(4-Fluoro-phenylcarbamoyl)-cyclopropanecarboxylic acid compound of Formula V (10.0 g, 1.0 equiv.), DMAP (13.68 g, 2.5 equiv.) and Dichloromethane (200 mL, 20.0 vol) under nitrogen atmosphere and the mixture was cooled to 0-5° C. Methane sulfonyl chloride (5.60 g6 g, 1.10 equiv.) was added by maintaining the temperature between 0 and 5° C. Now, 4-((6,7-dimethoxyquinolin-4-yl)oxy)aniline compound of Formula III (11.78 g, 0.90 equiv.) was added and the mixture was stirred at 0-5° C. After completion of the reaction, water (10.0 mL, 1.0 vol) was added and allowed to 25-30° C. Methanol (20 mL, 2.0 vol) was added and layers were separated. Organic layer was concentrated under reduced pressure, methanol (200 mL, 20.0 vol) was added and mass was stirred at between 25 to 30° C., filtered, washed with methanol (30 mL, 3.00 vol) and dried to obtain N-(4-(6,7-dimethoxyquinolin-4-yloxy)phenyl)-N′-(4-Fluorophenyl) cyclopropane-1,1-dicarboxamide compound of Formula II. Compound of Formula II was characterized by XRD as represented in FIG. 1. The characteristic peaks as characterized by an X-ray diffraction including peaks at 8.90, 10.60, 13.20, 13.70, and 21.80 degrees 2θ, +/−0.2 degrees, using Cu-Kα radiation.

### Example-12: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were charged with N-(4-(6,7-dimethoxyquinolin-4-yloxy)phenyl)-N′-(4Fluorophenyl) cyclopropane-1,1-dicarboxamide compound of Formula II (50.0 g, 1.00 equiv.), THF (600 mL, 12.0 Vol), water (50.0 mL, 1.0 Vol) and L-(−)-malic acid (16.0 g, 1.20 equiv.)the reaction mass was heated to 60-65° C. The above resultant solution was cooled to 25-30° C. and added into n-heptane (2000 mL, 40.0 Vol) at same temperature. The reaction mass was stirred for 1-2 h and filtered. washed with n-heptane (100.0 ml, 2.0 vol) and dried the solid at 50° C. under vacuum under vacuum to obtain Cabozantinib-S-Malate of Formula I. The solid obtained was characterized by XRD.

### Example-13: Preparation of N-(4-(6,7-dimethoxyquinolin-4-yloxy)phenyl)-N′-(4 Fluorophenyl) cyclopropane-1,1-dicarboxamide (Formula II)

To a reactor equipped with a stirrer, thermometer sensor and heating cooling system were added 1-(4-Fluoro-phenylcarbamoyl)-cyclopropanecarboxylic acid compound of Formula V (10.0 g, 1.0 equiv.), DMAP (13.68 g, 2.5 equiv.) and Dichloromethane (20.0 vol) under nitrogen atmosphere and the mixture was cooled to 0-5° C. Methane sulfonyl chloride (5.6 g, 1.10 equiv.) was added by maintaining the temperature between 0 and 5° C. Now, 4-((6,7-dimethoxyquinolin-4-yl)oxy)aniline compound of Formula III (11.78 g, 0.90 equiv.) was added and the mixture was stirred at 0-5° C. After completion of the reaction, water (1.0 vol) was added and allowed to 25-30° C. Methanol (20 ml, 2.0 vol) was added and layers were separated. Organic layer was concentrated under reduced pressure, methanol (200 ml, 20.0 vol) was added and mass was stirred at between 25 to 30° C., filtered, washed with methanol (30 ml, 3.0 vol) and dried to obtain N-(4-(6,7-dimethoxyquinolin-4-yloxy)phenyl)-N′-(4Fluorophenyl) cyclopropane-1,1-dicarboxamide compound of Formula II. The crystalline Compound of Formula II was characterized by XRD.

### Example-14: Preparation of N-(4-(6,7-dimethoxyquinolin-4-yloxy)phenyl)-N′-(4 fluorophenyl) cyclopropane-1,1-dicarboxamide, (2S)-hydroxybutanedioate (Formula I)

To a glass vessel equipped with a stirrer, condenser and a thermometer probe were charged with Formula II (20.0 g, 1.00 equiv.), methanol (500 mL, 25.0 Vol.), the reaction mass was heated to 40-45° C., filtered. Filtrate was added with Dichloromethane (500 mL, 25.0 Vol.) and L-(−) Malic acid (5.34 g, 1.00 equiv.). The solution was concentrated completely under vacuum at 40-45° C. to obtain Cabozantinib-S-Malate of Formula I as an amorphous solid. The solid obtained was characterized by XRD.

### Example-15: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were charged with Formula II (20.0 g, 1.00 equiv.), tetrahydrofuran (300 mL, 15.0 Vol.). To this solution, L-(−) Malic acid (5.34 g, 1.00 equiv.) dissolved in water (4.0 mL) was added. The reaction mass was heated to 40-45° C., filtered. The solution was concentrated completely under vacuum to obtain Cabozantinib-S-Malate of Formula I as an amorphous solid. The product obtained was characterized by XRD.

### Example-16: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a 500 mL glass vessel equipped with a stirrer, condenser and a thermometer probe were added N-(4-(6,7-dimethoxyquinolin-4-yloxy)phenyl)-N′-(4-Fluorophenyl) cyclopropane-1,1-dicarboxamide compound of Formula II (10.0 g, 1.00 equiv.), methanol (500 mL, 25 Vol.), Dichloromethane (500 mL, 25 Vol.), L-(−) Malic acid (2.74 g, 1.00 equiv.) and Eudragit-L-100® (6.37 g). The solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid dispersion. The solid obtained was characterized by XRD.

### Example-17: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a 500 mL glass vessel equipped with a stirrer, condenser and a thermometer probe were added N-(4-(6,7-dimethoxyquinolin-4-yloxy)phenyl)-N′-(4-Fluorophenyl) cyclopropane-1,1-dicarboxamide compound of Formula II (15.0 g, 1.00 equiv.), methanol (375 mL, 25 Vol.), Dichloromethane (375 mL, 25 Vol.), L-(−) Malic acid (4.01 g, 1.00 equiv.) and Eudragit-L-100® (19.0 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid dispersion.

### Example-18: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4-Fluorophenyl)cyclopropane-1,1-dicarboxamide compound of Formula II (5.00 g, 1.00 equiv.), methanol (125 mL, 25 Vol.), Dichloromethane (125 mL, 25 Vol.), L-(−) Malic acid (1.33 g, 1.00 equiv.) and Eudragit-L-100® (1.58 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid dispersion. The solid obtained was characterized by XRD.

### Example-19: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4Fluorophenyl) cyclopropane-1,1-dicarboxamide compound of Formula II (20 g, 1.00 equiv.), methanol (500 mL, 25 Vol.), Dichloromethane (500 mL, 25 Vol.), L-(−) Malic acid (5.32 g, 1.00 equiv.) and Eudragit-L-100® (3.79 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid dispersion. The solid obtained was characterized by XRD.

### Example-20: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4Fluorophenyl) cyclopropane-1,1-dicarboxamide compound of Formula II (20 g, 1.00 equiv.), methanol (500 mL, 25 Vol.), Dichloromethane (500 mL, 25 Vol.), L-(−) Malic acid (5.32 g, 1.00 equiv.) and Eudragit-L-100® (2.53 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid. The solid obtained was characterized by XRD.

### Example-21: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4-fluorophenyl)cyclopropane-1,1-dicarboxamide, (2S)-hydroxy butanedioate compound of Formula I (10 g, 1.00 equiv.), methanol (100 mL, 10 Vol.), Dichloromethane (100 mL, 10 Vol.), and PVP-K-30® (10.0 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid dispersion. The solid obtained was characterized by XRD.

### Example-22: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4Fluorophenyl) cyclopropane-1,1-dicarboxamide compound of Formula II (7.0 g, 1.00 equiv.), methanol (175 mL, 25 Vol.), Dichloromethane (175 mL, 25 Vol.), L-(−) Malic acid (1.87 g, 1.00 equiv.) and EthoCel-7CPS (8.87 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid dispersion. The solid obtained was characterized by XRD.

### Example-23: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4-fluorophenyl)cyclopropane-1,1-dicarboxamide, (2S)-hydroxy butanedioate compound of Formula I (2.00 g, 1.00 equiv.), methanol (50.0 mL, 25 Vol.), Dichloromethane (50.0 mL, 25 Vol.) and Hydroxy propyl methyl cellulose (HPMC-15C) (2.00 g). The resulting solution was concentrated under vacuum at 40-45° C. to obtain N-(4-(6,7-dimethoxyquinolin-4-yloxy)phenyl)-N′-(4fluorophenyl) cyclopropane-1,1-dicarboxamide, (2S)-hydroxy butanedioate compound of Formula I as a pasty mass.

### Example-24: Preparation of N-(4-(6,7-dimethoxyquinolin-4-yloxy)phenyl)-N′-(4 fluorophenyl)cyclopropane-1,1-dicarboxamide, (2S)-hydroxybutanedioate (Formula I)

To a glass vessel equipped with a stirrer, condenser and a thermometer probe were added N-(4-(6,7-dimethoxyquinolin-4-yloxy)phenyl)-N′-(4-Fluorophenyl)cyclopropane-1,1-dicarboxamide compound of Formula II (15 g, 1.00 equiv.), methanol (375 mL, 25 Vol.), Dichloromethane (375 mL, 25 Vol.), L-(−) Malic acid (4.01 g, 1.00 equiv.) and Eudragit-L-100® (0.38 g). The resulting solution was concentrated under vacuum at 40-45° C. to obtain N-(4-(6,7-dimethoxyquinolin-4-yloxy)phenyl)-N′-(4fluorophenyl)cyclopropane-1,1-dicarboxamide, (2S)-hydroxy butanedioate compound of Formula I as an amorphous solid. The solid obtained was characterized by XRD.

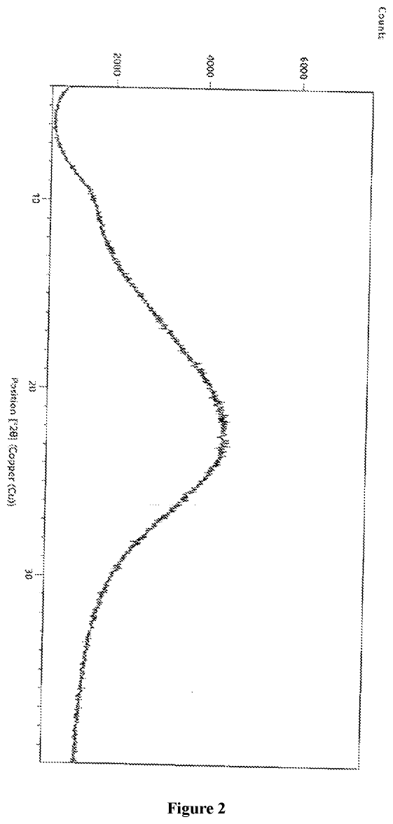

Compound of Formula II was characterized by XRD as represented in FIG. 2.

### Example-25: Preparation of Amorphous N-(4-(6,7-dimethoxyquinolin-4-yloxy)phenyl)-N′-(4-fluorophenyl)cyclopropane-1,1-dicarboxamide, (2S)-hydroxybutanedioate (Formula I)

To a 2 L glass vessel equipped with a stirrer, condenser and a thermometer probe were charged with a crystalline Formula I (50.0 g, 1.00 equiv.), THF (1000 mL, 20.0 Vol.) and methanol (250 mL, 5.0 Vol.), the reaction mass was heated to 40-45° C., filtered. The filtrate was concentrated under vacuum at 50-55° C. This was followed by dilution in DCM (1000 mL, 20.0 Vol.) and methanol (350 mL, 7.0 Vol.) at 25-30° C., filtered. Filtrate was concentrated completely under vacuum at 50-55° C. to obtain Cabozantinib-S-Malate of Formula I in amorphous form. It was characterized by XRD.

1.-7. (canceled)

8. A process for the preparation of N-(4-(6,7-dimethoxyquinolin-4-yloxy)phenyl)-N′-(4-Fluorophenyl) cyclopropane-1,1-dicarboxamide (compound of Formula II) or its malate salt (of Formula I) comprising the following steps:
a) activating 1-(4-Fluoro-phenylcarbamoyl)-cyclopropanecarboxylic acid (compound of Formula V) using a suitable sulfonyl chloride, optionally in presence of a suitable base in a suitable solvent,
b) adding 4-((6,7-dimethoxyquinolin-4-yl)oxy)aniline (compound of Formula III),
c) isolating N-(4-(6,7-dimethoxyquinolin-4-yloxy)phenyl)-N′-(4-Fluorophenyl) cyclopropane-1,1-dicarboxamide (compound of Formula II), and
d) optionally converting N-(4-(6,7-dimethoxyquinolin-4-yloxy)phenyl)-N′-(4-Fluorophenyl) cyclopropane-1,1-dicarboxamide (compound of Formula II) to its malate salt (of Formula I); or
a′) adding a suitable sulfonate chloride to a solution of 1-(4-Fluoro-phenylcarbamoyl)-cyclopropanecarboxylic acid (compound of Formula V) and 4-((6,7-dimethoxyquinolin-4-yl)oxy)aniline (compound of Formula III) in a suitable solvent,
b′) isolating N-(4-(6,7-dimethoxyquinolin-4-yloxy)phenyl)-N′-(4Fluorophenyl) cyclopropane-1,1-dicarboxamide (compound of Formula II); and
c′) optionally converting N-(4-(6,7-dimethoxyquinolin-4-yloxy)phenyl)-N′-(4-Fluorophenyl) cyclopropane-1,1-dicarboxamide (compound Formula II) to its malate salt (of Formula I).

9. The process for the preparation of N-(4-(6,7-dimethoxyquinolin-4-yloxy)phenyl)-N′-(4-Fluorophenyl) cyclopropane-1,1-dicarboxamide (compound of Formula II) or its malate salt (of Formula I) according to claim 8, comprising:
a) activating 1-(4-Fluoro-phenylcarbamoyl)-cyclopropanecarboxylic acid (compound of Formula V) using methane sulfonyl chloride, optionally in the presence of N,N-Dimethylamino Pyridine (DMAP) or N-methylimidazole (NMI) in dichloromethane,
b) adding 4((6,7-dimethoxyquinolin-4-yl)oxy)aniline (compound of Formula III),
c) isolating N-(4-(6,7-dimethoxyquinolin-4-yloxy)phenyl)-N′-(4-Fluorophenyl) cyclopropane-1,1-dicarboxamide (compound of Formula II), and
d) optionally converting N-(4-(6,7-dimethoxyquinolin-4-yloxy)phenyl)-N′-(4-Fluorophenyl) cyclopropane-1,1-dicarboxamide (compound of Formula II) to its malate salt (of Formula I).

10. The process for the preparation of N-(4-(6,7-dimethoxyquinolin-4-yloxy)phenyl)-N′-(4-Fluorophenyl) cyclopropane-1,1-dicarboxamide (compound of Formula II) or its malate salt (of Formula I) according to claim 8, comprising:
a) activating 1-(4-Fluoro-phenylcarbamoyl)-cyclopropanecarboxylic acid (compound of Formula V) using toluene sulfonyl chloride, optionally in the presence of N,N-Dimethylamino Pyridine (DMAP) or N-methylimidazole (NMI) in dichloromethane,
b) adding 4((6,7-dimethoxyquinolin-4-yl)oxy)aniline (compound of Formula III),
c) isolating N-(4-(6,7-dimethoxyquinolin-4-yloxy)phenyl)-N′-(4-Fluorophenyl) cyclopropane-1,1-dicarboxamide (compound of Formula II), and
d) optionally converting N-(4-(6,7-dimethoxyquinolin-4-yloxy)phenyl)-N′-(4-Fluorophenyl) cyclopropane-1,1-dicarboxamide (compound of Formula II) to its malate salt (of Formula I).

11. (canceled)

12. The process for the preparation of N-(4-(6,7-dimethoxyquinolin-4-yloxy)phenyl)-N′-(4-Fluorophenyl) cyclopropane-1,1-dicarboxamide (compound of Formula II) or its malate salt (of Formula I) according to claim 8 comprising:
a) adding methane sulfonyl chloride to a solution of 1-(4-Fluoro-phenylcarbamoyl)-cyclopropanecarboxylic acid (compound of Formula V) and 4-((6,7-dimethoxyquinolin-4-yl)oxy) aniline (compound of Formula III) in dichloromethane and a suitable base,
b) isolating N-(4-(6,7-dimethoxyquinolin-4-yloxy)phenyl)-N′-(4-Fluorophenyl) cyclopropane-1,1-dicarboxamide (compound of Formula II), and
c) optionally converting N-(4-(6,7-dimethoxyquinolin-4-yloxy)phenyl)-N′-(4-Fluorophenyl) cyclopropane-1,1-dicarboxamide (compound of Formula II) to its malate salt (of Formula I),
wherein the suitable base is selected from N,N-Dimethylamino Pyridine (DMAP) and N-methylimidazole (NMI).

13. The process for the preparation of N-(4-(6,7-dimethoxyquinolin-4-yloxy)phenyl)-N′-(4-Fluorophenyl) cyclopropane-1,1-dicarboxamide (compound of Formula II) or its malate salt (of Formula I) according to claim 8 comprising:
a) adding toluene sulfonyl chloride to a solution of 1-(4-Fluoro-phenylcarbamoyl)-cyclopropanecarboxylic acid (compound of Formula V) and 4-((6,7-dimethoxyquinolin-4-yl)oxy) aniline (compound of Formula III) in dichloromethane and a suitable base,
b) isolating N-(4-(6,7-dimethoxyquinolin-4-yloxy)phenyl)-N′-(4Fluorophenyl) cyclopropane-1,1-dicarboxamide (compound of Formula II), and
c) optionally converting N-(4-(6,7-dimethoxyquinolin-4-yloxy)phenyl)-N′-(4-Fluorophenyl) cyclopropane-1,1-dicarboxamide (compound of Formula II) to its malate salt (of Formula I),
wherein the suitable base is selected from N,N-Dimethylamino Pyridine (DMAP) and N-methylimidazole (NMI).

14. A compound selected from the group consisting of a compound Formula X, compound Formula XI, compound Formula XIV, and a compound Formula IV:

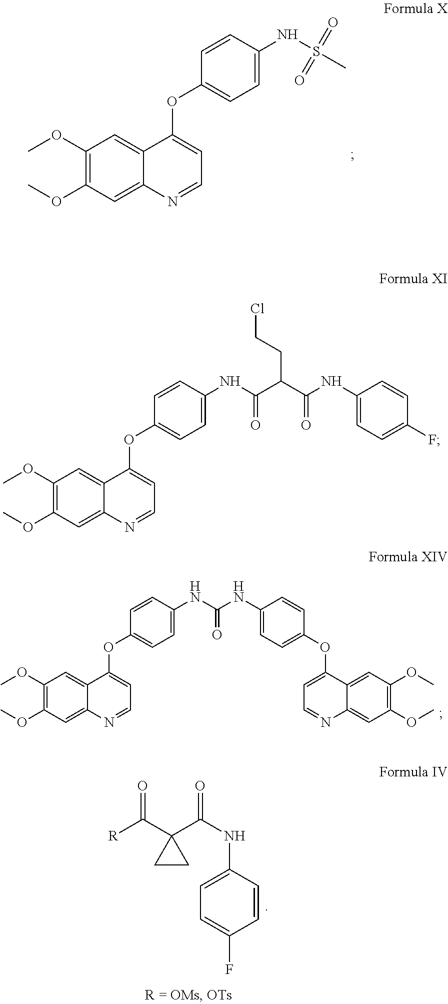

15. (canceled)

16. (canceled)

17. The process according to claim 8, which is substantially free of any one of the impurities selected from the group consisting of compounds Formula X, Formula XI, and Formula XIV.

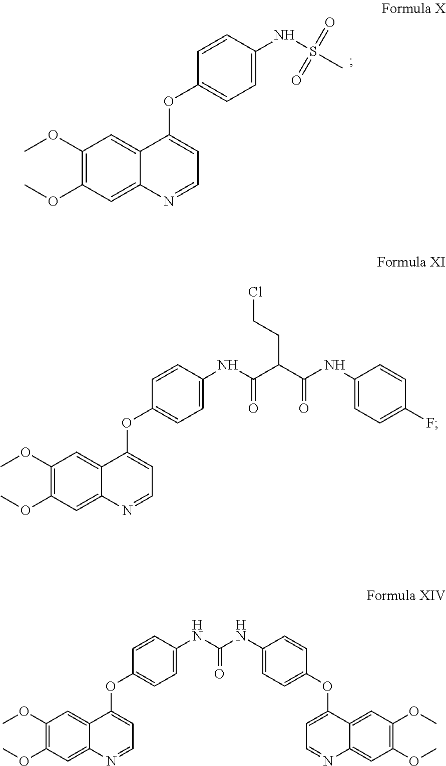

18. The process according to claim 8, wherein in step (a) or (a′) the suitable solvent is selected from dichloromethane, dichloroethane, THF and acetonitrile.

19. The process according to claim 8, wherein the suitable sulfonyl chloride is selected from the list of methane sulfonyl chloride, p-toluene sulfonyl chloride, 4-chlorobenzylsulfonyl chloride, 2-chlorobenzylsulfonyl chloride, and 4-nitrophenyl sulfonyl chloride.

20. The process according to claim 8, wherein the suitable base is an organic base selected from N,N-dimethylamino pyridine (DMAP) and N-methylimidazole (NMI).
